(19) United States
(12) Patent Application Publication
(10) Pub. No.: US 2008/0274234 A1
(43) Pub. Date: Nov. 6, 2008

(54) PROCESSING COCOA BEANS AND OTHER SEEDS

(76) Inventor: Carter Robert Miller, Ilheus (BR)

Correspondence Address: FISH & RICHARDSON P.C., P.O BOX 1022, Minneapolis, MN, 55440-1022, US

(21) Appl. No.: 12/111,591

(22) Filed: Apr. 29, 2008

Related U.S. Application Data
(60) Provisional application No. 60/915,313, filed on May 1, 2007.

(51) Int. Cl. A23G 1/02; A23L 1/36; A23G 1/00; A23P 1/00

(52) U.S. Cl. 426/45; 426/489; 426/44; 426/472; 426/466; 426/443; 426/629

(57) ABSTRACT

A method of treating seeds includes piercing a multiplicity of seeds such that shells of a majority of the seeds are pierced, aerating the pierced seeds, and reducing a water content of the pierced seeds. Another method of treating seeds includes placing a bulk quantity of seeds in a container, forming a mass of seeds and liquid in the container, sealing the container to create a substantially closed environment inside the container, and fermenting the mass in the sealed container. Another method of treating seeds includes placing a multiplicity of pierced seeds in a ventilated enclosure, forcing air through the enclosure such that the seeds are exposed to the air, and mixing the seeds.

### CROSS-REFERENCE TO RELATED APPLICATIONS

This application claims the benefit under 35 U.S.C. §119(e)(1) of U.S. provisional application 60/915,313, filed May 1, 2007, which is incorporated by reference herein in its entirety.

### TECHNICAL FIELD

This invention relates to methods of processing seeds from the fruits of the tree Theobroma cacao L., known as cocoa beans, and other seeds, including species and varieties of, and hybrids among and between, the species of the genera Theobroma and Herrenia. This invention further relates to the resulting products of such methods.

### BACKGROUND

The seed of the fruit of the tree, Theobroma cacao L., is generally known as the cocoa bean. Cocoa beans are widely processed to derive chocolate and cocoa products, and for extraction of nutrients, flavor compounds, and phytochemicals contained in cocoa. Generally, the combined processes of fermenting and drying cocoa beans to produce dry, green cocoa beans, known as curing, is requisite to obtain flavor precursors. The flavor precursors, upon roasting, create the distinctive aromas and taste compounds instilling cocoa and cocoa-derived products with chocolate flavor.

Traditional cocoa bean curing tends to result in various degrees of non-homogeneity among the dry, green cocoa beans used as a principal ingredient in specialty chocolate and confectionery, as well as in other food, cosmetic, and medical industries. High levels of heterogeneity among dry green cocoa bean can deleteriously affect processing of green cocoa, by costly processes to overcome these deficiencies, and can result in a less flavorful, nutritious, and/or useful product. Similar curing processes, when applied to other seeds, including species and varieties of, and hybrids among and between, the species of the genera Theobroma and Herrenia, are thought to result in similar heterogeneity.

### SUMMARY

According to one aspect of the invention, a method of treating seeds includes piercing a multiplicity of seeds such that shells of a majority of the seeds are pierced, aerating the pierced seeds, and reducing water content of the pierced seeds.

In some embodiments, the seeds include cocoa beans. The majority of the seeds are unfermented at the time of piercing or fermented before piercing. Piercing the seeds includes forming an opening in each shell (testa, also referred to as skin before drying and hull when a dry seed) of the majority of the seeds. Each opening has an opening area of between about 0.5 and 15 mm². Piercing the multiplicity of seeds may include forming an opening in the shell, and cotyledon, of the majority of the seeds. Piercing the multiplicity of seeds may include forming one or more openings in the majority of seeds with one or more needles, with a jet of fluid, droplets of enzymes or acids, and/or with electromagnetic radiation.

In some embodiments, a method of treating seeds includes curing the multiplicity of seeds. Piercing the multiplicity of seeds may occur before curing the multiplicity of seeds. The multiplicity of seeds has an average water content of at least about 10 wt % during piercing. Reducing the water content of the pierced seeds may include reducing an average water content of the pierced seeds to less than about 10 wt %, less than about 8 wt %, or to between about 6 and 8 wt %. A method of treating seeds may include roasting the pierced seeds.

Another aspect of the invention includes a bulk quantity of treated seeds, in which a majority of the treated seeds have pierced shells, and an average water content of the treated seeds is less than about 10 wt %.

In various embodiments, the treated seeds have an average water content less than about 8 wt %, or between about 2 and 8 wt %. The pierced shells may have one or more openings in each pierced shell. The openings may be substantially uniform. The openings may extend through the shell and into a cotyledon of the majority of the seeds. The openings are typically surrounded by intact shell. A portion of the cotyledon proximate the opening is exposed to atmosphere. The majority of the seeds may be dry green cocoa beans or roasted cocoa beans.

According to another aspect of the invention, a method of treating seeds includes placing a bulk quantity of seeds in a container, forming a mass in the container, sealing the container to create a substantially closed environment inside the container, and fermenting the mass in the sealed container. The mass includes the bulk quantity of seeds and liquid.

In some embodiments, the seeds include cocoa beans. Fermenting the mass includes alcoholic fermentation, alcohol-induced metabolic stress response, up regulation and down regulation (expression) of genes, nucleic acids, and proteins, programmed cell death, proteolysis and autolysis of cells that lead to the inviability of the seed embryo and death of the seeds. A method of treating seeds may include mixing the mass in the sealed container, controlling an amount of oxygen in the container, and/or controlling an amount of carbon dioxide in the container. A method of treating seeds may include piercing the bulk quantity of seeds, or piercing the seeds before placing the seeds in the container.

In some embodiments, the liquid includes a sucrose-containing solution. In some embodiments, the liquid includes juice and pulp from cacao fruit. A majority of the weight of the liquid consists of the juice and pulp. A method of treating seeds includes monitoring a temperature within the sealed container, controlling a temperature within the sealed container, and/or maintaining a temperature within the sealed container at less than about 35° C. A method of treating seeds may include controlling a pressure inside the sealed container, controlling a pH of the liquid, controlling a titratable acidity, and/or monitoring dissolved gases in the liquid.

In some embodiments, a majority of the seeds are at least partially submerged in the liquid. Visible radiation may be inhibited from entering the sealed container during fermentation. Gas may be selectively added to the sealed container during fermentation. A method of treating seeds may include adding microorganisms, enzymes, and/or one or more additives to the liquid. Additives may be selected from the group including sugars, preservatives, and stabilizers.

According to yet another aspect of the invention, a method of treating seeds includes placing a multiplicity of fermented seeds in a ventilated enclosure, forcing air through the enclosure such that the seeds in the enclosure are exposed to the air, and mixing the seeds.

In some embodiments, the seeds include cocoa beans. A method of treating seeds includes placing the seeds on a tray and placing the tray in a cabinet. The enclosure may be a food dehydrator. The method may include monitoring a temperature of the air, a temperature inside the enclosure, and/or a relative humidity inside the enclosure. A temperature of the air may be between about 22° C. and about 32° C., at least about 40° C., or in a range between about 40 and 80° C. Mixing may include manually mixing and/or mechanically mixing. The method may include reversing a direction of the forced air. In some embodiments, a majority of the seeds are pierced. In some embodiments, a majority of the pierced seeds are pierced in one or more locations.

In one aspect, a bulk quantity of fermented, dry, unroasted cocoa beans has an average titratable acidity of less than about 1.1 mL of 0.1 N NaOH per gram of cocoa beans. In some embodiments, an average free ammonia of the bulk quantity is less than about 500 ppm, less than about 100 ppm, or less than about 50 ppm.

In some cases, the bulk quantity was made by the process comprising fermenting the bulk quantity for at least one week, and the bulk quantity has an average fermentation index less than about 1.0. In some cases, the bulk quantity was made by the process comprising fermenting the bulk quantity for at least two weeks, and the bulk quantity has an average fermentation index less than about 1.0. In some cases, the bulk quantity was made by the process comprising fermenting the bulk quantity for at least three weeks, and the bulk quantity has an average fermentation index less than about 1.1. In some cases, the bulk quantity was made by the process comprising fermenting the bulk quantity for at least four weeks, and the bulk quantity has an average fermentation index less than about 1.25.

In some embodiments, the bulk quantity was made by the process comprising alcoholic fermentation. The total oxygen radical absorbance capacity, in some cases, is at least about 400 μmole Trolox equivalent per gram of cocoa beans. The water-soluble oxygen radical absorbance capacity is about 100 times greater than the lipid-soluble oxygen radical absorbance capacity. In some cases, the fermentation factor of the bulk quantity is about 400, that is, substantially all of the cocoa beans are brown.

In another aspect, a bulk quantity of fermented, dry, unroasted cocoa beans, has been fermented for at least about 4 weeks, and the bulk quantity has a fermentation index of less than about 1.2 and a fermentation factor of about 400. In some embodiments, the fermentation index of the bulk quantity is less than about 1.1.

In some embodiments, the bulk quantity has been fermented for at least about 3 weeks, and the bulk quantity has a fermentation index of less than about 1.1 or less than about 1.0.

In some embodiments, the bulk quantity has been fermented for at least about 2 weeks, and the bulk quantity has a fermentation index of less than about 1.0 or about 1.0, or less than about 0.9 and greater than about 0.7.

In some embodiments, the bulk quantity has been fermented for at least about 1 week, and the bulk quantity has a fermentation index of less than about 0.9, or less than about 0.8 and greater than about 0.6.

In some cases, the titratable acidity of a sample of the bulk quantity is less than about 1.2, 1.1, or 1.0 ml 0.1 N NaOH per gram of the sample.

In one aspect, a bulk quantity of seeds is treated according to a process including piercing a multiplicity of seeds such that shells of a majority of the seeds are pierced, aerating the pierced seeds, and reducing a water content of the pierced seeds.

In another aspect, a bulk quantity of seeds is treated according to a process including placing a bulk quantity of seeds in a container, forming a mass including the bulk quantity of seeds and liquid in the container, sealing the container to create a substantially closed environment inside the container, and fermenting the mass in the sealed container.

In another aspect, a bulk quantity of seeds is treated according to a process including placing a multiplicity of pierced seeds in a ventilated enclosure, forcing air through the enclosure such that the seeds are exposed to the air, and mixing the seeds.

In another aspect, a method of producing a bulk quantity of fermented, dry cocoa beans includes piercing a multiplicity of cocoa seeds such that shells of a majority of the cocoa seeds are pierced, aerating the pierced cocoa seeds, fermenting the pierced cocoa seeds, and reducing water content of the pierced cocoa seeds to produce the bulk quantity of the fermented, dry cocoa beans.

In another aspect, a method of producing a bulk quantity of fermented, dry cocoa beans includes placing a multiplicity of cocoa seeds in a container, forming a mass including the multiplicity of cocoa seeds and liquid in the container, sealing the container to create a substantially closed environment inside the container, fermenting the mass in the sealed container, and reducing water content of the fermented mass to produce the bulk quantity of the fermented, dry cocoa beans.

In another aspect, a bulk quantity of the fermented, dry cocoa beans is made by the process comprising the steps of (a) piercing a multiplicity of cocoa seeds such that shells of a majority of the cocoa seeds are pierced; (b) aerating the pierced cocoa seeds; (c) fermenting the pierced cocoa seeds; and (d) reducing water content of the pierced cocoa seeds to produce the bulk quantity of the fermented, dry cocoa beans.

In another aspect, a bulk quantity of the fermented, dry cocoa beans is made by the process comprising the steps of: (a) placing a multiplicity of cocoa seeds in a container; (b) forming a mass including the multiplicity of cocoa seeds and liquid in the container; (c) sealing the container to create a substantially closed environment inside the container; (d) fermenting the mass in the sealed container; and (e) reducing water content of the fermented mass to produce the bulk quantity of the fermented, dry cocoa beans.

Following processing as described above, the cracked pieces of the germ and cotyledons, known as cocoa beans—those pieces of the interior of the bean that remain after separation of shell or bran—have improved homogeneity, consistent fermentation and browning, good nutrition, pleasant flavor, preserved phytochemical content, and other desirable quality parameters important for food, medical, and cosmetic applications. Generally, flavor and aroma development important in taste perception may be more highly controlled and varied as preferred by the processor and tailored to the local situation as characteristics such as quality of fruits harvested, varieties used for processing, and quality and duration of fermentation and aeration vary over time. Alcoholic and glycolytic fermentation may impart unique taste, flavor, aroma, nutritional, pharmacological and medicinal characteristics due to increased ethanol contents and decreased acidity and acetic acid contents and lower temperatures of the liquid medium that has contact with the cocoa bean interior during and after bean death. Low-temperature curing may avert changing of phases of cocoa lipids from solid phase to liquid phase, improve the permeability of seeds, and/or limit lipid breakdown and free fatty acid production, while increasing aeration to non-lipid components of the seed. Physical, chemical, and flavor characteristics of cocoa butter and cocoa solids may be enhanced as a result of improved isolation of seed constituents during curing.

Pierced seeds may undergo more precisely controlled reactions within the seed interior environment including, but not limited to, anaerobic, reducing, aerobic, and/or low dissolved CO₂, enzymatic processes (e.g., hydrolytic and proteolytic processes), and non-enzymatic biochemical processes. Proteins such as, but not limited to, seed storage protein albumin, globulin, prolamine, and glutelin may undergo proteolytic reactions more readily and to a greater extent in pierced seeds. Protein breakdown products such as, but not limited to, polypeptides and amino acids as well as other nitrogenous compounds, such as ammonia and nitrate, may undergo oxidation, condensation reactions (tanning), volatilization, or methylation more readily and to a greater extent in pierced seeds. Pierced seeds dehydrate or dry more readily and with less energy input than traditionally treated seeds (for instance, cocoa beans). Additionally, the pierced shell acts to improve heat and moisture transfer to the interior of the bean during curing, drying, pre-roasting, and roasting. Wet ‘dutching’ processes may proceed more efficiently due to increased penetration of compounds such as dissolved salts and enzymes to the bean interior. In-shell roasting is improved, due to improved efficiency of energy transfer to the bean interior as well as improved control of bean and nib moisture content and air pressure during roasting, and less energy is wasted heating the otherwise highly impermeable shell. In-shell roasting proceeds more readily and with improved evenness of roast throughout seeds that have been pierced, lessening over-roasting of small seeds and under-roasting of large seeds. As well, efficiency of cracking and winnowing is improved as the shell more readily separates from the nib upon cracking. Improved fermentation and action of enzymatic reactions such as cellulases promote more substantial cell wall breakdown and facilitate processes such as roasting, dutching and grinding of nibs. Reduced acidity of the product lessens the necessity for and/or shortens the time period required to achieve desired dry and wet conching of the cocoa mass.

### BRIEF DESCRIPTION OF THE DRAWINGS

FIG. 1 depicts a flow diagram of steps in a method of treating seeds.

FIG. 2 depicts a schematic view of a fermentation chamber

FIG. 3 depicts a schematic view of a dehydration chamber.

FIGS. 4A-4B depict steps in a seed piercing process.

FIG. 5A depicts a cross-sectional view of an intact seed with a pierced shell.

FIG. 5B depicts a cross-sectional view of an intact seed with a pierced shell and a pierced cotyledon.

FIG. 5C depicts a cross-sectional view of an intact seed with a pierced shell and two pierced cotyledons.

FIG. 5D depicts a cross-sectional view of an intact seed with a continuous opening extending from one portion of the shell, through the cotyledons, and through a second portion of the shell.

FIGS. 6A-6F depict steps in a continuous seed piercing process.

FIG. 6G depicts a schematic view of a pierced seed.

FIG. 7A is a photograph of fermenting cocoa beans during an early stage of fermentation.

FIG. 7B is a photograph of fermenting cocoa beans during a later stage of fermentation.

FIG. 7C is a photograph showing fermented cocoa bean cotyledons.

FIG. 7D is a photograph showing the interior of a peeled, fermented cocoa bean.

FIG. 7E is a photograph showing another view of the interior of a cut, fermented cocoa bean.

FIG. 8 is a photograph showing fermented cocoa bean cotyledons after condensation.

FIG. 9 is a photograph showing an exterior of a pierced cocoa bean.

FIG. 10A is a photograph showing cocoa bean cotyledons during early stage aeration.

FIG. 10B is a photograph showing cocoa bean cotyledons during late stage aeration.

FIG. 10C is a photograph showing cocoa bean cotyledons after full aeration.

FIG. 11A is a photograph showing openings in a shell of a pierced, dry, green cocoa bean.

FIG. 11B is a photograph showing an interior of a pierced, dry, green cocoa bean.

FIG. 11C is a photograph showing dry, green cocoa bean cotyledons.

Like reference symbols in the various drawings indicate like elements.

### DETAILED DESCRIPTION

As used herein, “fruit,” “pulp,” “seeds,” “shells,” “cotyledons,” etc., generally refer to those portions derived from fruits of the trees of the tree specie Theobroma cacao L., often referred to in the art as cocoa pods, pulp, beans, skins or hulls, and meat or nibs, respectively. While examples herein refer to cocoa beans or seeds, it is to be understood that the methods described below generally refer to other seeds as well, including seeds of fruits from species and varieties of, and hybrids among and between the species of the genera Theobroma and Herrenia, that would undergo or would benefit from processing that includes transportation of fluid across a membrane or layer (for instance, shell) of the seed. “Treating,” as used herein, generally refers to preparing seeds from harvested fruits for ingestion or topical use, including cosmetic, pharmaceutical and medicinal uses.

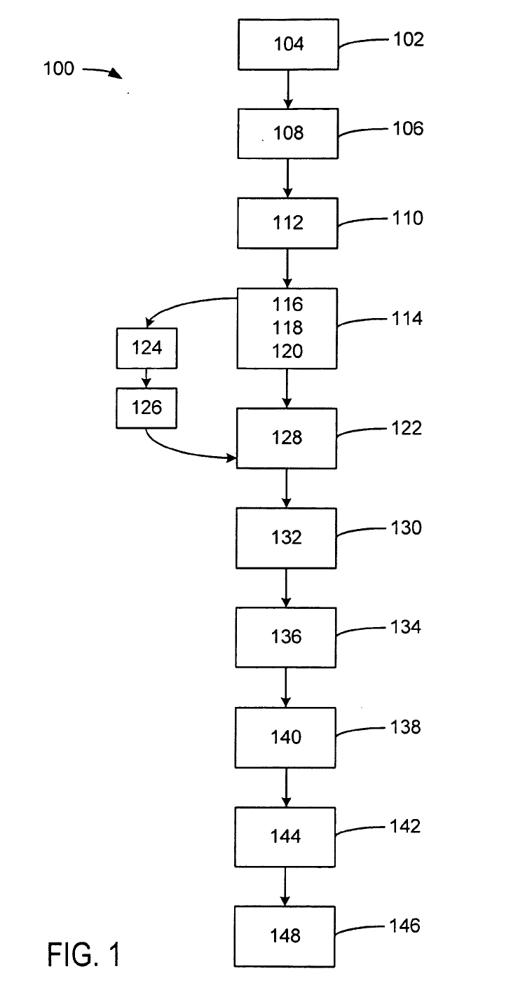

FIG. 1 depicts a flow chart of process 100 for treating a multiplicity or bulk quantity of seeds. As used herein, a “multiplicity” or “bulk quantity” generally refers to a number of seeds that are being processed together for use or sale. After harvesting 102, fruit 104 is cleaned and inspected 106. Cleaned and inspected fruit 108 is de-husked or opened 110 to yield wet, juice and pulp-encased seeds 112 (wet cacao). De-husking or opening 110 may include removing placental material. Wet seeds with juice and pulp may be frozen or fresh frozen and later thawed.

Juicing and depulping 114 of parenchymatous tissue that surrounds and adheres to the exterior of seeds 116 may be achieved by mechanically scraping pulp 118 from the shells (testa), resulting in the release of sweet liquid fruit juices 120 and separation of fibrous pulp from seeds 116. In some cases, juicing may be achieved by centrifugation or pressing. In some cases, two or more seeds that are adhered together may be separated. In some cases, depulped seeds may be visually analyzed or otherwise distinguished by their pigmentation or relative content of other distinguishable components, via internal spectral analysis of the seeds. Seeds and seed pieces of variously distinguished traits in the cotyledons, such as polyphenolic concentration or type, may be graded and separated by grade. Separated graded seeds may be further processed separately or recombined at any point in the process.

The resulting pulp 118 and juice 120 may be filtered (or not) and maintained separately or recombined with the seeds 116. Wet seeds 116 are placed in a clean (for instance, sterilized), food-safe container for fermentation 122. Liquid 124, including fruit juice 120 and pulp 118, is placed in the container before, during, or after placement of wet seeds 116 in the container. Placental material may be included along with pulp 118. Pulp 118 and juice 120 may account for a majority of the weight of liquid 124. “Fermenting mass” 126 generally refers to seeds 116 and fermenting liquid 124, which may include pulp 118 from fruit 104.

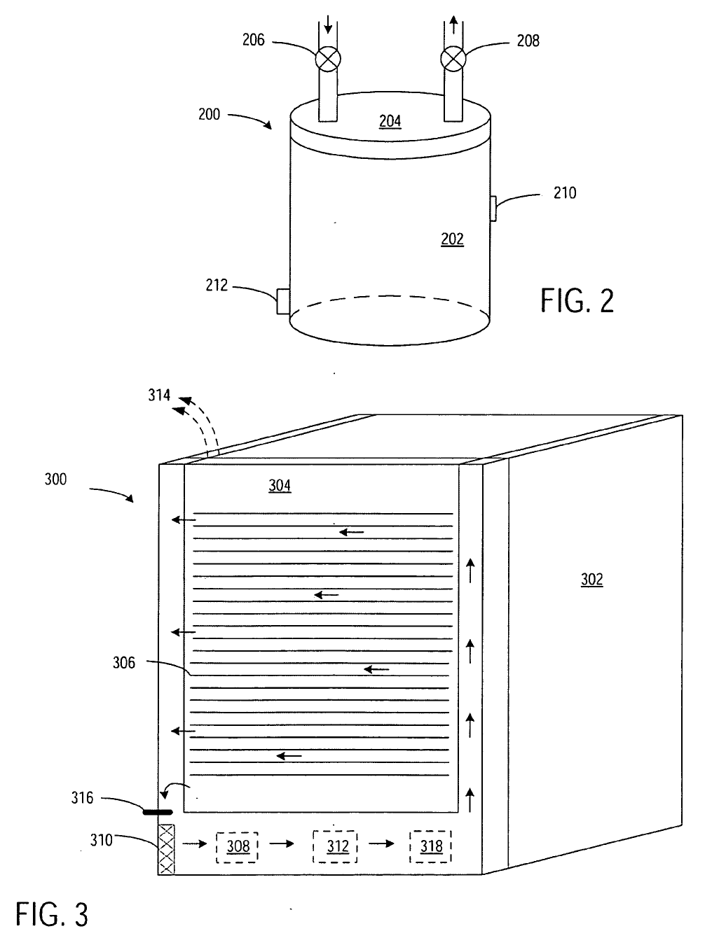

Fermentation 122 may be achieved by a variety of methods, including traditional mound, box, or controlled chamber fermentation common in the art and generally characterized by production of ‘sweatings,’ with high dissolved oxygen tension in the fermenting mass, high acetic acid generation, high titratable acidity, low pH, and high fermentation temperatures or at fermentation temperatures that increase as fermentation progresses, and generally little or no care given to hygiene and sanitation of materials. In some embodiments, fermentation 122 takes place in a container such as container 200, depicted in FIG. 2, for fermentation of a multiplicity or bulk quantity of seeds. Container 200 may be clean and dry or sterilized or autoclaved and of any size, shape, and/or material as desired to contain a fermenting mass in a food-safe environment. For instance, container 200 may be an inox container of any size and shape, a glass carboy, a plastic bucket, or a polyethylene terephthalate drum. The material for container 200 may be chosen to be substantially opaque to inhibit exposure of the fermenting mass to visible radiation.

Container 200 (such as depicted in FIG. 2) may allow fermentation in a substantially closed environment. In some embodiments, fermentation may begin before container 200 is sealed or before the container becomes a substantially closed environment. Limiting the amount of oxygen present in the container promotes anaerobic fermentation of the fermenting mass, which may inhibit acetic acid bacteria, production of acetic acid, and subsequent uptake of acetic acid by the seeds. The fermentation may be alcoholic fermentation facilitated by microbiological, enzymatic, and/or biochemical activity. Fermentation may commence for a significant period prior to sealing of the container. The container can be covered by a gas permeable material or filter (e.g., cloth, filter paper, gas permeable film) or loosely fitting cover.

Container 200 includes body 202 and lid 204. Lid 204 is sealed to body 202 to create a closed environment during fermentation. Container 200 may include an airlock or one or more valves (for instance, check valves) to selectively allow introduction or removal of fluid. As shown in FIG. 2, valves 206, 208 are coupled to lid 204 of container 200. In some embodiments, valves 206, 208 are coupled to body 202 of container 200. In other embodiments, one or more valves may be coupled to the lid of container, and one or more valves may be coupled to the body of the container.

Valve 206 may be coupled to a reagent source (for instance, a gas cylinder) to allow introduction of gas into the container during fermentation. In an example, oxygen gas may be added to a fermenting mass in container 200 to facilitate fermentation. Valve 208 may allow removal of fluid from container before, during, or after fermentation. For instance, air from container 200 may be evacuated through valve 208 after the seeds and liquid have been placed in the container to promote anaerobic fermentation. Valve 208 may be used to allow selective removal or exhaust of fermentation products such as carbon dioxide. In some embodiments, volatile compounds released from the fermenting mass may be collected, identified, and/or quantified. Volatile compounds may include, for instance, flavor compounds.

In some embodiments, container 200 may include one or more sealable ports 210 in body 202 and/or lid 204 of the container. Ports 210 may be used to allow monitoring of the fermentation process, including monitoring of the fermenting mass, liquid, and/or products of fermentation. For instance, a probe inserted through port 210 may allow control and/or monitoring of properties including, but not limited to, temperature, pH, pressure, titratable acidity, or combinations thereof. In some cases, chemical compounds in the container (for instance, ethanol, acetic acid, polyphenols, flavonoids) may be identified and/or quantified. The conversion of carbohydrates to alcohol, production of alcohol, brix (dissolved sugars) level, dissolved alcohol content, or combinations thereof may be monitored.

During fermentation, a majority of the seeds may be submerged in the liquid in container 200, such that the submerged seeds are each surrounded by the liquid. During fermentation, it may be desirable to mix, agitate, turn, or stir the fermenting mass. In some cases, the cap may be pushed down. This may be achieved manually, for instance, after removing lid 204 while maintaining a positive gas pressure in the body of the container to inhibit influx of the atmosphere, or mechanically, by inserting an implement through port 210 into the fermenting mass.

A temperature within the container may be monitored and/or controlled during fermentation. It may be desirable to maintain a temperature of less than about 40° C., less than about 35° C., less than about 30° C., or less than about 20° C. in container 200 during fermentation. Temperature control may be achieved, for instance, by a heat pump and a thermostat operatively coupled to the container, or the container may be water jacketed. In some embodiments, it may be desirable to selectively elevate a temperature within the container for a limited time to, for instance, effect a flavor change, kill off microbes, denature enzymes, or pasteurize the contents of the container. Following the temperature elevation, the mass may be allowed to cool, or may be actively cooled, before further processing, such as addition of enzymes.

During fermentation, a pH and/or titratable acidity of the fermenting mass or liquid may be monitored and/or controlled. The pH of the fermenting mass, generally expected to be acidic, may be increased by the addition of, for instance, calcium carbonate. Before fermentation begins, a pH of the pulp may be around 3. The pH of the fermenting mass may rise during fermentation. A basic pH may indicate the presence of one or more contaminants in the container.

During fermentation, an amount of one or more gases dissolved in the fermenting mass or liquid or otherwise in the container (for instance, above the fermenting mass) may be monitored and/or controlled. Monitored gases may include, but are not limited to, oxygen, carbon dioxide, and ammonia. These and other gases may be selectively added or removed as desired to enhance fermentation.

Other additives may be provided to the fermenting mass before or after body 202 is sealed with lid 204. Additives may include, but are not limited to, microorganisms, enzymes, carbohydrates (sugars), preservatives, and stabilizers.

Microorganisms may be added by inoculation or introduced by spontaneous aerial or surface contact contamination before or during fermentation and may include yeasts, for instance, Saccharomyces spp., S. cerevisiae, S. cerevisiae var. chevalieri, Candida spp. Kloackera apis, Kluyveromyces spp., lactic acid bacteria, and acetic acid bacteria, and/or combinations thereof. Microorganisms with high alcohol tolerance and conversion efficiency may be desirable.

Enzymes may be added before or during fermentation and may include, but are not limited to, pectinases. In an example, ULTRAZYM®, a pectinase available from Novozymes A/S (Bagsvaerd, Denmark), is added to the fermenting mass.

Carbohydrates (for instance, sucrose, fructose, glucose, maltose, or fruit juice) may be added before or during fermentation as a source of energy. Preservatives (for instance, potassium metabisulfate) may be added as desired.

Alcoholic fermentation of seeds in a closed, monitored, and/or controlled environment may inhibit production of acetic acid and subsequent uptake of acetic acid by the seeds that generally occurs during traditional aerobic fermentation, in which pulp, along with “sweatings,” are allowed to exit from the fermenting mass. Controlled fermentation, resulting in alcohol- and/or lactic acid-induced death of the seeds (including the cotyledons and other portions of the seeds), may also advantageously result in more homogeneous seeds after fermentation. In a controlled fermentation process, lysing and/or plumping relating to seed death and increased moisture content of the seeds may occur at higher relative frequency and at a lower temperature and lower acetic acid concentration than traditional seed fermentation, also promoting homogeneity. Homogeneity may be assessed, in some cases, by visual inspection of the physical appearance and color of the cotyledons. When cut open after fermentation but before drying, a uniformly wet or plumped seed interior of a dead seed having color that may range from creamy white to pink and purple may be more desirable than the dry and lusterless appearance of unfermented cotyledons before fermenting or after incomplete or inadequate fermentation. In some cases, partial or incomplete fermentation, in which the seeds are only partially plumped or without any noticeable plumping, may be advantageous.

Controlled fermentation of the fermenting mass in container 200 may occur over a period of one or more days, one or more weeks, or up to four months, or longer. Alcoholic fermentation may proceed at a more gradual rate, more slowly, over longer periods of time than traditional box or mound fermentations. A duration of fermentation may be chosen to affect desirable flavor characteristics of the seeds. In the case of longer fermentations, it may be advantageous to reduce the head space above the fermenting mass to limit penetration of gases, such as oxygen, to the fermenting mass. One or more seeds may be removed from the container and inspected or tested for desirable properties (for instance, plumping or homogeneity). When the seeds have been desirably fermented, lid 204 is removed from body 202 to open container 200, and the fermenting (or now fermented) mass is removed from the container. In some embodiments, exit 212, which may include a valve or other scaled access, allows removal of liquid or fermented mass from container 200 with the aid of gravity.

As depicted in FIG. 1, wet fermented mass 128 may be condensed 130 to condense (concentrate) and partially dry pulp 118 adhered to the seeds 116. Condensation 130 may be carried out under reduced light conditions. For instance, condensation may occur in the absence of visible light, or with filtered or reduced visible light, or in the presence of natural light, with or without the presence of ultraviolet and/or infrared radiation. Condensation 130 may reduce moisture in wet fermented mass 128, resulting in a moist fermented mass 132 that is semi-wet, “tacky wet,” moist or dry to the touch, while the seed interiors (cotyledons) may remain wet or moist. Condensation 130 may occur over a time period of about less than 4 hours, 4 to 12 hours, or 12 to 48 hours or more.

In some embodiments, condensation may be achieved in a pressurized environment or in a partial vacuum. A partial vacuum may be desirable for desiccating the fermented mass. Condensation and/or aeration may achieved with convective airflow or other methods known in the art such as utilizing a drying platform, rotary drum, or fluid bed drier. In an example, a fermented mass may be placed in a food dehydrator with a stacked tray design and horizontal forced airflow. In some cases, vacuum microwave drying (VMD) is used, for example, to inhibit decomposition of antioxidants during the drying process. Drying methods, including VMD, can be initiated following fermentation, depulping, condensation, or perforation.

FIG. 3 depicts a schematic view of an embodiment of a dryer. In an example, dryer 300 includes cabinet 302 and door 304. In this embodiment, cabinet 302 is aluminum. Door 304 may have a see-through portion to allow visual monitoring of the seeds during condensation and/or aeration. Trays 306 with openings in a bottom portion of the trays are supported in cabinet 302. Trays 306 may have dimensions of, for example, about 1 m×0.5 m. The openings may be woven screen made from aluminum wires of 0.7 mm diameter stretched both lengthwise and widthwise across the interior of the tray at about 7 mm intervals. Cabinet 302 holds trays 306 in a vertical array with a vertical spacing of, for example, 55 mm.

Electric fan 308 may draw air through air intake 310 past heater 312 and over trays 306. Air intake 310 may be a regulated air intake. Air that enters through air intake 310 may be filtered to substantially remove contaminants, such as dust, microorganisms, or viruses from the air. Airflow may be regulated continuously or non-continuously. In some embodiments, heater 312 may be, for example, a natural gas burner. Air may exit the dryer through exhaust 314 and/or through open door 304. Exhaust 314 may be a regulated exhaust. Recirculation plate 316 may be fully adjustable to facilitate control of recirculation of heated air or to bypass recirculation of air. Dryer 300 may have a thermostat 318 coupled to heater 312 to control a temperature of air in cabinet 302. Air temperature may be maintained at or below 50° C. by use of heater 312.

Precise atmospheric control in dryer 300 may be achieved by controlling a relative humidity in the dryer and monitoring oxygen and carbon dioxide content in the dryer. Moisture may be added upwind of the seeds, for instance, by providing a humidifier or jets of fine mist or smaller particles to create a fog surrounding seeds in dryer 300. Maintaining a desired humidity will inhibit drying of the seeds before completion of desired wet aerobic reactions. In some embodiments, dryer 300 includes a relative humidity sensor, a carbon dioxide sensor, and/or an oxygen sensor located upwind and/or downwind of the seeds. Dryer 300 may also include a carbon dioxide scrubber and/or an oxygen inlet upwind of the seeds.

A quantity of fermented mass 128 may be placed on tray 306 and distributed substantially uniformly and condensed 130. A fermented mass, the thickness of about 20 mm, may be desirable. Fermented mass 128 may be manipulated (spread or mixed) manually or mechanically. Trays 306 may be manually or mechanically rotated (for instance, by 180°) to reverse the direction of airflow across fermented mass 128 and to even out drying and/or condensation of the pulp. Trays 306 may be removed from cabinet 302 and placed on a stable surface while the seeds and pulp are mixed and redistributed over the tray. Mixing of the fermented mass and/or rotation of the trays may occur at intervals of about 2 to 3 hours or as necessary to promote an even rate of evaporative moisture loss from the pulp while maintaining moist seed interiors. The mixing process may decrease clumping of the pulp, reduce adhesion of the pulp to the seeds, and inhibit adhesion of seeds to each other as the pulp condenses and dries. Condensation 130 is continued and moisture content is reduced until the moist, fermented mass of seeds 132 can be handled or stored with minimal or no adhesion of the seeds to surfaces or to each other.

As depicted in FIG. 1, seeds 116 may be pierced or perforated 134 following condensation of the pulp or prior to or during condensation 130. As used herein, “pierce” generally refers to forming an opening in a seed, while leaving the portion of the seed surrounding the opening substantially intact. “Intact” generally refers to unitary or whole. A pierced seed may be a perforated seed. A “perforated” seed refers to a seed pierced in two or more locations to form two or more openings. The openings may be substantially uniform in size and/or shape. An area of the openings may range between about 0.5 and 15 mm². In some cases, an area of the opening may be smaller than 0.5 mm² or larger than 15 mm². The openings may have shapes including, but not limited to, circular, rectangular, oval, or star-shaped.

Seeds may be pierced in a variety of methods, such as piercing with a solid object, piercing with a fluid jet, piercing with droplets of enzymes or acids, piercing with electromagnetic radiation, or combinations thereof. Piercing with a solid object may include piercing with a sharpened metal cylinder. The sharpened metal cylinder may be, for instance, a solid or hollow needle. Piercing with a fluid jet may include, but is not limited to, piercing with an air jet, a water jet, or a jet of gas including, but not limited to, argon, nitrogen, oxygen, carbon dioxide, and combinations thereof. Piercing with droplets may include, but is not limited to, piercing with liquid droplets of cellulases or pectinases or acids such as hydrochloric acid or hydrogen peroxide or combinations there of. Piercing with electromagnetic radiation may include piercing with visible laser radiation.

Pierced or perforated seeds facilitate the transport of fluid and dissolved gasses from the outer environment across the shell to the interior of the seed (cotyledons, embryo) and transport of fluid and dissolved gasses from the interior of the seed across shell to the exterior environment while allowing the seed as well as the shell to remain substantially intact. Piercings or perforations of shell and seed interior may act in similar fashion or be likened to pores. Pierced or perforated shells have a significantly increased porous nature relative to non-pierced shells, while the shell remains substantially intact surrounding the piercings, and the seed interior is not directly exposed to and remains substantially protected from the outer environment. Shape, size, positioning, and number of piercings may be chosen to impart selected flavor and/or nutritional characteristics to seeds. A seed may include openings of various depths, including one or more openings that extend through an entire thickness of the seed and one or more openings that extend partially through the seed. Openings in seeds may be used to facilitate transport of any fluid across the shell and into the cotyledons. Fluids may be chosen, for instance, to improve oxidizing reactions such as browning and tanning, to preserve the seed (or inhibit oxidation of the seed), or to add flavoring to the seed. Pierced seeds may allow uniform penetration of the seed by heat or fluid (originating from inside or outside of the seed), resulting in more homogenous cured or roasted seeds.

As depicted in FIG. 1, wet, semi-wet or moist, “tacky wet”, or partially dried seeds 116 may be pierced 134 after fermentation 122 to yield pierced seeds 136. In some cases, piercing 134 may occur before fermentation 122 or condensation 130, or before or after any step in the treatment process depicted in FIG. 1. Piercing 134 may occur while a water content of a seed inhibits cracking of the shell during piercing. For example, piercing 134 may occur when a water content of a seed is greater than about 40 wt %, greater than about 20 wt %, or greater than about 10 wt %. By way of example, seed piercing is described below as related to piercing of fermented seeds with needles.

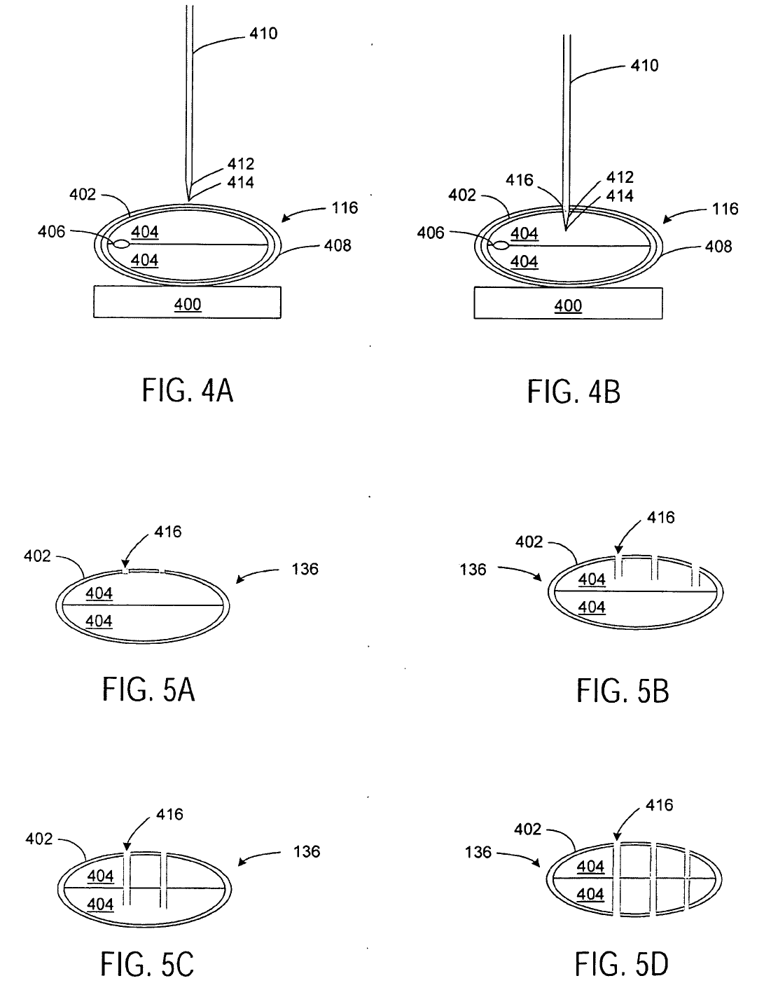

A process of piercing seeds is depicted in FIGS. 4A and 4B. Seed 116 may be positioned on surface 400. Surface 400 may have any composition or texture designed to promote stationary positioning of seed 116. That is, surface 400 may inhibit translation and/or rotation of seed 116 with respect to the surface. For instance, surface 400 may include a polymeric memory material that holds seed 116 in place when a downward force is exerted on the seed.

As shown in the cross-sectional view of seed 116 in FIG. 4A, the seed includes shell (testa) 402, cotyledons 404, and germ (embryo and root radical) 406. Condensed pulp 408 may be adhered to at least a portion of shell 402. Needle 410 is shown positioned above seed 116. In an example, needle 410 may have a length ranging from about 45 mm to about 65 mm or longer and a diameter ranging from about 0.5 mm to about 2 mm. Needle 410 may include tapered end 412 and point 414. In some embodiments, tapered end 412 and/or point 414 may be a cutting edge or tip. Tapered end 412 may be from about 0.5 mm to about 4 mm in length. Needle 410 may be fabricated of any strong, non-corrosive, food-safe material, including stainless steel (for instance, inox). As depicted in FIG. 4B, needle 410 may be advanced through pulp 408 and shell 402 of seed 116 to form opening 416 in pierced seed 136.

As depicted in the cross-sectional view of pierced seed 136 in FIG. 5A, piercing may be limited to shell 402, while cotyledons 404 remain substantially unpenetrated. As depicted in FIG. 5B, piercing may include forming opening 416 in shell 402 and cotyledon 404 proximate the opening in the shell. As depicted in FIG. 5C, piercing may include forming opening 416 in shell 402 and more than one cotyledon 404 in seed 136. As depicted in FIG. 5D, piercing may include forming opening 416 through an entire thickness of seed 136, such that the opening extends from a first location on shell 402, through one or more cotyledons 404, and through a second location on the shell.

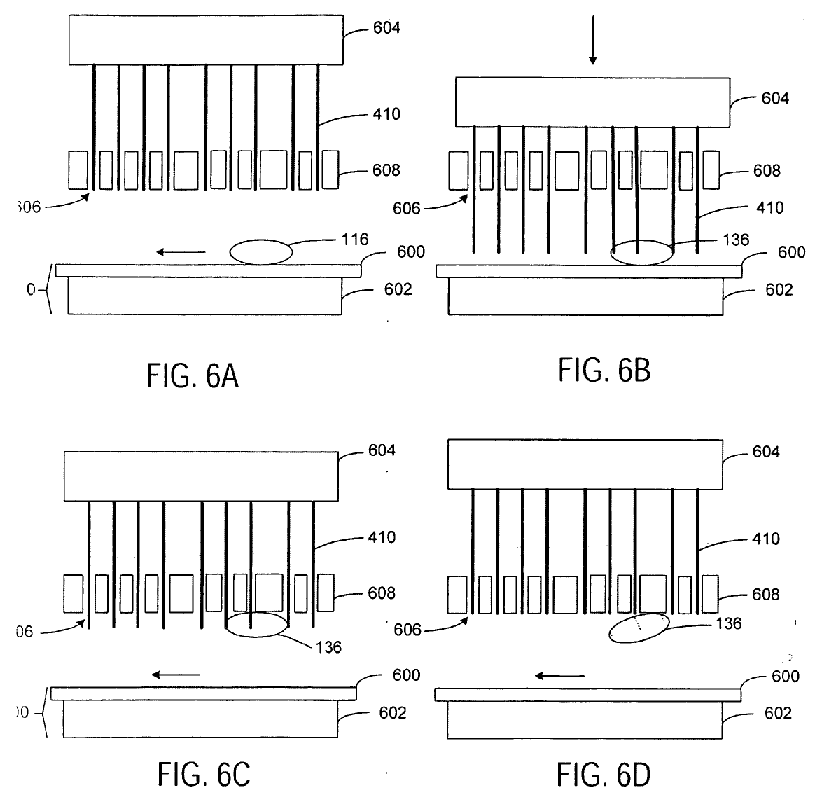

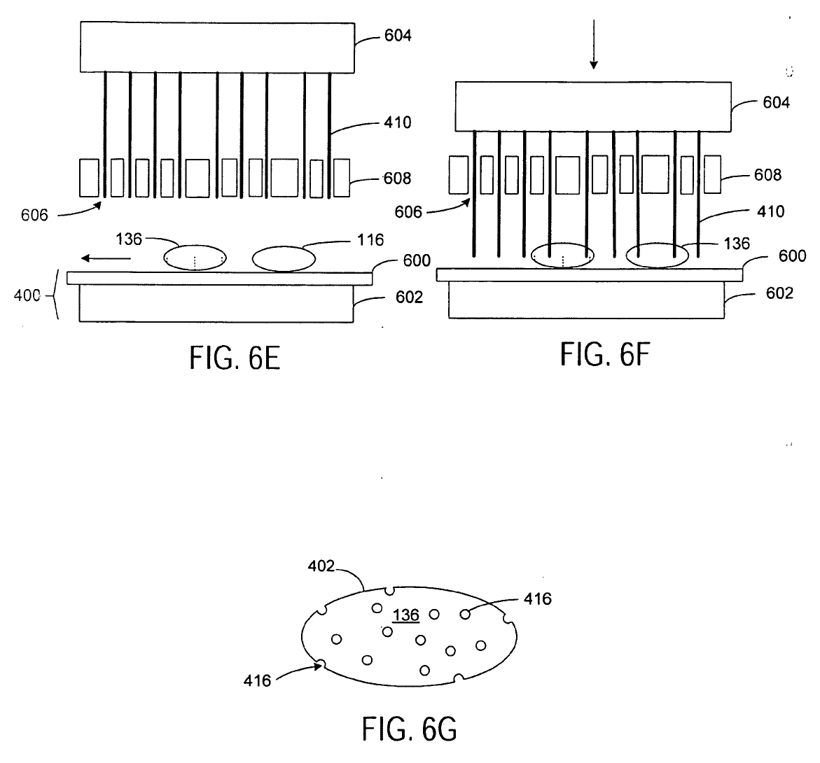

In one embodiment, steps in a continuous process for piercing a multiplicity of seeds are depicted in FIGS. 6A-6F. Seeds 116 are placed in a hopper and are conveyed on a food-grade flexible belt system in a dispersed single layer onto surface 400. Surface 400 includes conveyor 600 and platform 602. Needles 410 may be held securely in plate 604 above conveyor 600 and lowered vertically downward through guide 608. In an example, guide 608 is positioned about 55 mm above conveyor 600. Needles 410 may be inserted and retracted one or more times, for instance 5 to 10 times or more, such that the lowered needles pierce the fermented pulp (if any) and one or more portions of seeds 116. A height of plate 604 and/or guide 608 and/or a length of the needles 410 may be chosen to inhibit contact of tip 414 of the needle with surface 400.

A multiplicity of needles 410 may be coupled to plate 604 define a piercing region with an area of about 100×300 mm. Needles 410 may be arranged, for instance, in 10 rows of 30 needles each, with about 10 mm between needles in a row along a width of the piercing region and about 8 mm between rows along the length of the piercing region. Seeds 116 on conveyor 600 may be pierced as they pass through the piercing region. Pierced seeds 136 may be collected from conveyor 600. Pierced seeds 136 may be reloaded into the hopper and passed through the piercing region one or more additional times, such that a majority of the seeds are sufficiently pierced yet remain intact. In some cases, motion of the conveyor may be stopped or reversed to allow additional piercing of seeds.

As depicted in FIG. 6A, seed 116 is on conveyor 600 above platform 602. Needles 410 are coupled to plate 604 and positioned through openings 606 in guide 608. Plate 604 is retracted, such that needles 410 do not extend beyond a lower surface of guide 608. As plate 604 is lowered, depicted in FIG. 6B, needles 410 pierce seeds 116 to form pierced seeds 136. As plate 604 is raised, depicted in FIG. 6C, pierced seeds 136 may be lifted off conveyor 600 by needles 410. If pierced seeds 136 are retained on needles 410 during retraction of the needles, the seeds are released from needles 410 after pierced seeds 136 contact guide 608, and fall back to conveyor 600.

Needles 410 may be of the same or different lengths to allow formation of openings of the same or different dimensions. Needles 410 may be advanced and retracted more than once, or repeatedly. For example, needles 410 may be advanced and retracted in substantially uniform intervals as conveyor 600 moves seeds 116 in a plane perpendicular to a longitudinal axis of the needles 410, as depicted in FIG. 6D.

The orientation of pierced seeds 136 on conveyor 600 in FIG. 6E may differ from the orientation of seeds 116 on the conveyor in FIG. 6A, exposing unpierced portions of seeds 136 to needles 410. Pierced seeds 136 and unpierced seeds 116 advance on conveyor 600, as depicted in FIG. 6E. As needles 410 are again extended through guide 608 as depicted in FIG. 6F, seeds 136 are pierced again by the needles, forming an additional one or more openings 416 at least partially through the seeds. Pierced, intact seeds 136 exit the piercing region on conveyor 600. With a multiplicity of seeds 116 on conveyor 600, multiple seeds 136 are pierced substantially simultaneously, such that openings 416 are formed in a majority of the seeds.

FIG. 6G depicts a schematic view of pierced seed 136 with openings 416. Shell 402 is intact. Openings 416 provide surface area inside the cotyledons for exchange of fluids involved in chemical processes (such as enzymatic browning and non-enzymatic browning) and physical processes (such as drying) throughout the seed, while allowing the cotyledons to remain in the protective shell. Openings 416 act as channels to allow fluid to flow from outside of the shell, from an interior portion of the shell, and/or from an exterior portion of the cotyledons toward an interior portion of the cotyledons.

Controlled movement of fluid through openings in a pierced seed allows chemicals such as polyphenols and enzymes that are concentrated in the exterior of the cotyledons, in the shell, and on the outside of the shell to infiltrate, by osmosis, mass flow, or other means, the cotyledon interiors, or wick inward in an oxidizing front, to achieve substantially uniform distribution of these chemicals throughout the cotyledons. These polyphenols and enzymes are important to precursor formation (for instance, precursors for chemicals that enhance flavor content and advantageous pharmacological, medicinal, and cosmetic characteristics). Thus, pierced (or perforated seeds) allow improved homogeneity of treated seeds, and leaving the shell intact allows desirable substances from the shell, the shell exterior (for instance, condensed pulp), and the shell interior as well as those from the exterior of the cotyledon to enter, osmotically migrate, or infuse the cotyledons during treating. In addition, piercing may be achieved without producing broken bits of shell that contaminate the cotyledons and require removal during subsequent processing.

In contrast, seeds with shells that have been cracked, broken, scored, crushed, scraped, winnowed, or cut do not benefit from controlled movement of fluid from an exterior of a seed toward an interior of the seed. Removing the shell from portions of the seed reduces or eliminates the flow of beneficial substances from the outer portion of the seed (shell exterior or interior) toward the inner portion of the seed. Cutting, crushing, cracking, or similar processing of a seed may separate portions of a seed and inhibit flow from one portion of a seed to another. Thus, interior portions of a cotyledon, after such processing, may have the same exposure to the environment as exterior portions of the cotyledon, both without the benefit of possible infusion of substances from the shell or other portion of the seed.

A seed that has been deshelled or otherwise cracked, broken, scored, crushed, scraped, winnowed, or cut has increased exposure of cotyledon exteriors and interiors to the environment, and less contact of the cotyledon exteriors with the shell. This exposure promotes drying or oxidizing of portions of all exposed surfaces, and does not allow controlled osmotic wicking from a seed exterior toward a seed interior. With increased exposure of seed interiors caused by cutting, crushing, etc. and removal of shell from at least portions of the seed, wicking of beneficial substances from the seed exterior toward the seed interior is reduced or eliminated, and beneficial substances from an exterior of a seed may not permeate an entire seed in uniform manner. Thus, development of desirable characteristics that result from these beneficial substances is absent, incomplete, or reduced.

In addition, pressure exerted on a seed during cracking, breaking, scoring, crushing, scraping, winnowing, or cutting (for instance, between rollers) may result in cotyledon cell damage, and the compression caused by pressure may inhibit uniform fluid exchange in the cotyledons. Furthermore, cracking, breaking, scoring, crushing, or cutting may result in pieces of shell mixed in with or implanted in the cotyledons (nibs), requiring later removal.

As depicted in FIG. 1, pierced seeds may undergo aeration 138. Aeration 138 may be achieved similarly to condensation 130. That is, seeds 136 may be aerated in dryer 300 depicted in FIG. 3. In some embodiments, trays 306 may be rotated at intervals of about 4 to 12 hours or about 8 to 24 hours for about 1 to 14 days, or longer as needed. Convective or radiant dehydration, or a combination thereof, using positive pressure and/or convective airflow, may be used to dehydrate cured, pierced seeds to produce dried, cured seeds. Aeration 138 may be regarded as a second fermentation step, in which aerobic fermentation (non-oxygen-limiting) with gas exchange and controlled relative humidity, light, temperature, etc., results in significant and homogeneous oxidation (enzymatic and non-enzymatic browning) of the cotyledons, embryo, and root radical.

Following and/or during aeration 138, seeds 140 may undergo dehydration 142. Dehydration 142 may be achieved similarly to condensation 130 or aeration 138 in dryer 300 depicted in FIG. 3. A temperature during dehydration may be maintained at or below ambient temperature, at about 45° C. or less, about 50° C. or less, or about 60° C. or less. A temperature during dehydration may be maintained from about 50° C. to 60° C., about 60° C. to 70° C., or about 70° C. to 80° C. Relative humidity (RH) may be maintained above about 90%, from about 80-90%, or below about 80%. Trays may be rotated at 1 or 2 hour intervals for a duration of 2 to 4 or more hours or until moisture in the seeds is reduced to 6-8 wt %, characteristic of dry, fermented or cured (“green” or unroasted) seeds.

Moisture content of seeds such as cocoa beans may be estimated by touch and or by listening for characteristic sounds associated with cracking seeds with a known moisture content. Moisture content may be assessed quantitatively with other methods, including drying methods, infrared detection (MM710 Food Gauge, available from NDC Infrared Engineering USA.; Irwindale, Calif.), NMR detection (Spin Track, Resonance Systems; Mary El, Russian Federation), and electrical response (G-7 Grain Moisture Meter, Delmhurst Instrument Co.; Towaco, N.J.).

Following dehydration 142, as depicted in FIG. 1, the dry, fermented or cured seeds 144 (in some embodiments, dry, green cocoa beans), may be processed 146 to yield processed seeds 148. Processing may include roasting (including, for instance, wetting, reconstituting, and pasteurizing of dry, cured seeds 144). About 15 L of dry, cured seeds 144 may be placed in a steel rotary drum roaster with a volume of about 30 L. The roaster may be rotated at about 50 rpm. Roasting temperatures may range between about 120° C. to 150° C., and roasting times may range from about 15 to 20 minutes up to about 45 to 90 minutes. Processing 146 may also include, but is not limited to, winnowing, dutching, grinding, conching, refining, and pressing or extracting of cocoa butter and milling of the pressed cake to cocoa powder.

In some processes, volatile flavor compounds released from fermenting, aerating, drying, dried or roasted seeds may be collected. The volatile compounds may be condensed before or after collection. Phytochemicals including, but not limited to, flavonoids, isoflavones, and phytosterols, may be extracted from roasted, or dried, or wet fermented, or frozen, or fresh frozen, or freeze dried seeds or portions thereof by ethanol/methanol extraction, supercritical CO₂ extraction, or other extraction methods known in the art.

Further processing of roasted seeds by cracking and winnowing produces nibs (cotyledons) and shell, a bran product, high in soluble and insoluble fiber, having a pleasant chocolate flavor and aroma and antioxidant qualities due, in part, to polyphenolic and other phytochemical compounds resulting from the method of treating described herein.

The cracked pieces of the cotyledons (known as cocoa nibs in the case of cocoa beans) and germ—those pieces of the interior to the bean that remain after separation of shell or bran—have improved homogeneity, consistent fermentation and browning, good nutrition, pleasant flavor and aroma, preserved phytochemical content, and other desirable quality parameters important for food, pharmacological, medical, and cosmetic applications.

Process 100 in FIG. 1 depicts a method for treating seeds including harvesting 102, cleaning and inspection, 106, opening 110, depulping 114, fermenting 122, condensing 130, piercing 134, aerating 138, dehydrating 142, and processing 146. Steps in process 100 may be added, omitted, or performed in an order other than that depicted in FIG. 1. For instance, another embodiment of process 100 includes harvesting 102, cleaning and inspection, 106, opening 110, fermenting 122, depulping 114, piercing 134, aerating 138, and dehydrating 142. Another process 100 includes harvesting 102, cleaning and inspection, 106, opening 110, depulping 114, fermenting 122, condensing 130, piercing 134, dehydrating 142, and processing 146. Yet another process 100 includes harvesting 102, cleaning and inspection, 106, opening 110, fermenting 122, depulping 114, piercing 134, and dehydrating 142. Still another process 100 includes harvesting 102, opening 110, fermenting 122, depulping 114, (with or without piercing 134), (with or without aerating 138), and dehydrating. Process 100, without piercing 134, results in non-perforated seeds.

Various processes allow for tailoring of taste (flavor and aroma) and/or nutritional properties of the seeds. Depulping directly after fermenting allows a shortened processing time while still resulting in a substantially uniform product. Enzymatic browning results if a temperature during aeration is kept under a temperature required for enzyme denaturation (for instance, in a range of about 50° C. to 65° C.). Drying above an enzyme denaturation temperature allows non-enzymatic browning. The dry, green seeds may then undergo processing that includes enzymatic dutching.

In some embodiments, traditionally fermented wet seeds that have been depulped, or those that are partially dried, may be condensed, pierced, aerated, and dried in a mechanical dehydrator or on traditional drying surfaces using processes described herein. In some embodiments, after the removal of fermented wet seeds and pulp from a fermentation container, such as that depicted in FIG. 2, seeds and pulp, together or after separation by depulping, may be frozen or freeze dried and used for extractions of nutrients, flavor compounds, and phytochemicals.

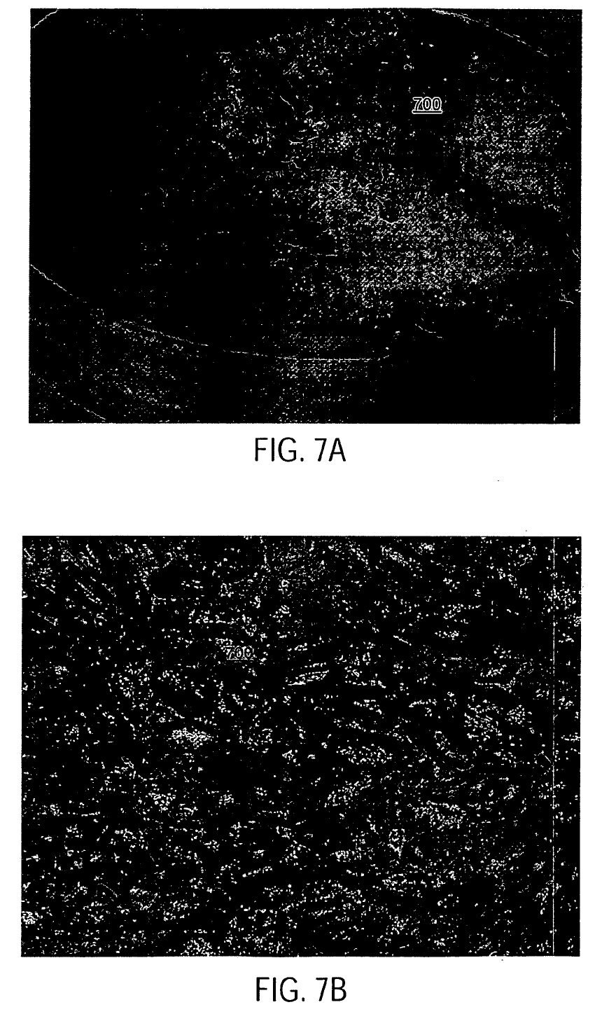

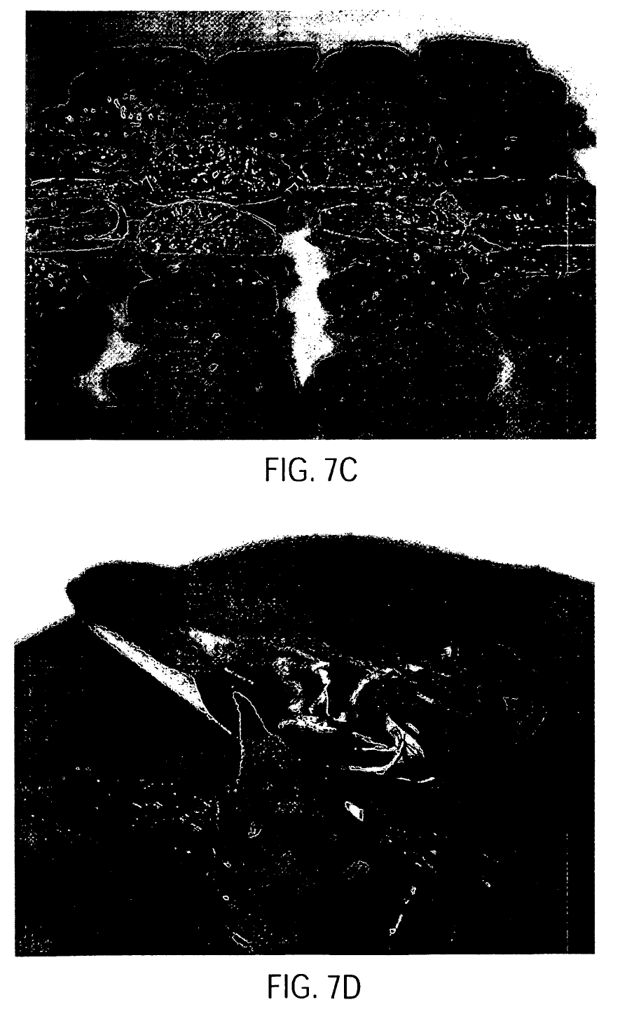

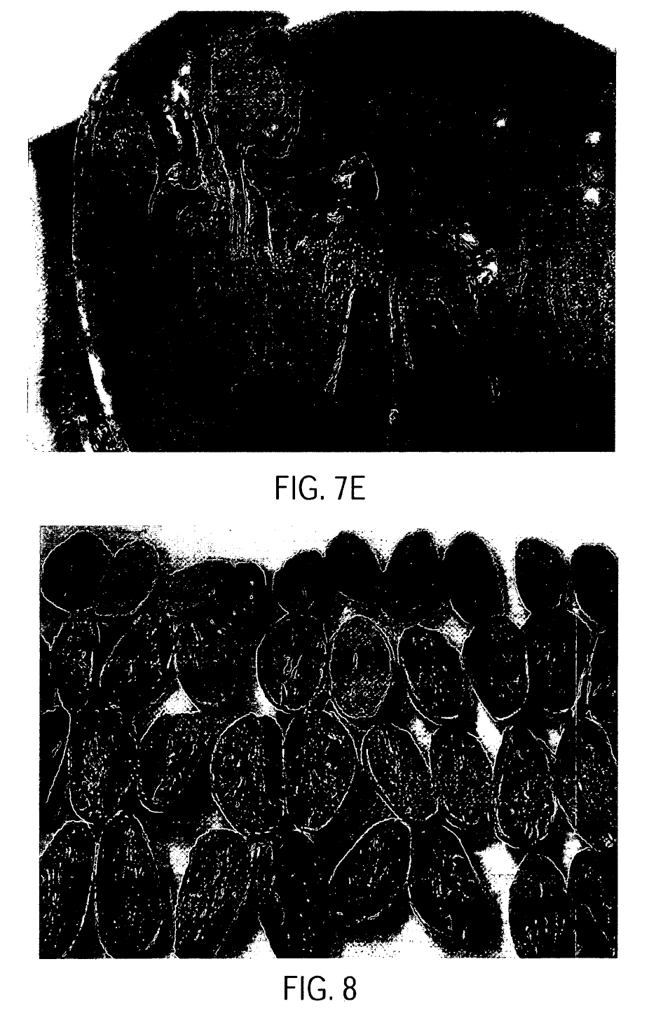

Cocoa beans treated by one or more steps in process 100 have been examined for desirable physical characteristics, including homogeneity. The cocoa beans chosen for the photographs in FIGS. 7-11 were selected randomly from a multiplicity of treated cocoa beans and cut open for visual inspection.

FIG. 7A is a photograph of a fermenting mass relatively early in fermentation 122. FIG. 7B is a photograph of a fermenting mass later in the fermentation process. As shown in FIGS. 7A and 7B, must 700 changes from white or cream-colored to a purplish color during fermentation as polyphenols from the cocoa beans exits through the shell and enters the must. FIG. 7C is a photograph of cocoa beans with exposed cotyledons after fermentation 122. The cotyledons exhibit a substantially uniform glossy, plumped appearance caused by penetration of liquid from the must to the seed interior due to cell lysing upon seed death. Seed coloration varies from creamy white to purple depending on polyphenolic content of the seed.

FIG. 7D is a photograph showing the interior of a peeled (shell removed), fermented cocoa bean. FIG. 7E is a photograph showing the interior of a cut, fermented cocoa bean. Purple pigmentation of the cotyledons is noticeably darker along the exterior of the fermented cotyledons. This dark purple pigmentation indicates the presence of flavor precursor compounds including polyphenols. Piercing of cocoa beans after fermentation promotes osmosis or wicking of these flavor precursor compounds toward the cotyledon interior during further processing, such as aeration. In the absence of wicking toward the cotyledon interior, the darker purple exterior region becomes dark brown during aeration and/or drying, resulting in a less homogenous bean (darker toward the exterior, lighter toward the interior) with less desirable properties, including less desirable flavor characteristics.

In the case of cocoa beans that have been deshelled or otherwise cracked, broken, scored, crushed, scraped, winnowed, or cut rather than pierced, interior browning may be less enzymatic and have less polyphenols as well as higher concentrations of non-converted polyphenols in outer portions of the seed and lower concentrations in portions of the seed exposed to the environment. This higher concentration is evident as a dark brown outer portion of the cotyledon toward an exterior of the seed, in contrast with lighter portions of the cotyledon toward an interior of the seed. The darker portion is characterized by increased bitterness, while the lighter portion is more astringent, and the seed has relatively weak and/or unevenly distributed (unbalanced) flavor precursor development.

FIG. 8 is a photograph of cocoa bean cotyledons after fermentation 122 and condensation 130.

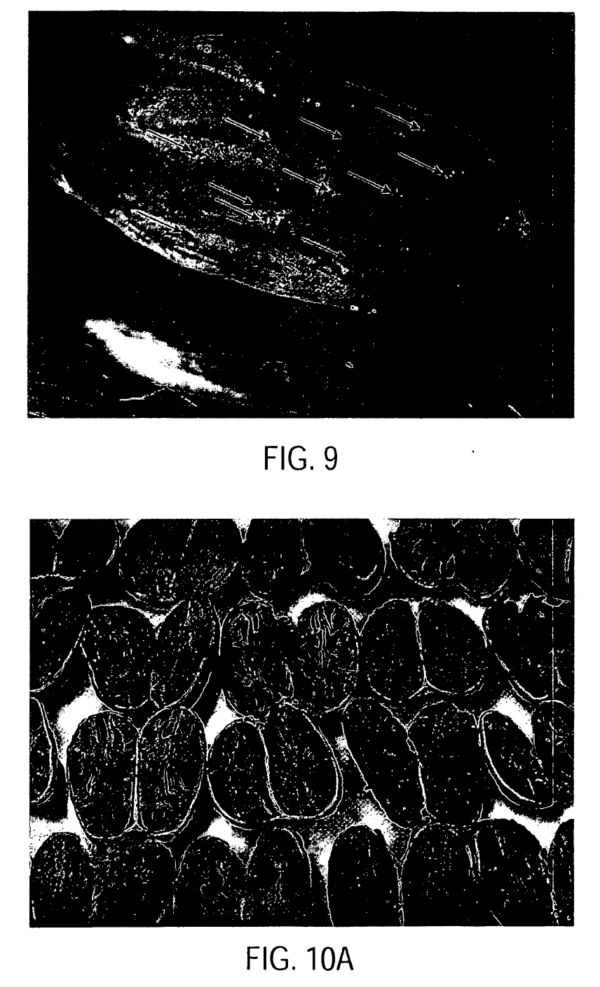

FIG. 9 is a photograph of an exterior of a cocoa bean that has undergone depulping 114, fermentation 122, and piercing 134. The arrows indicate some of the openings in the cocoa bean.

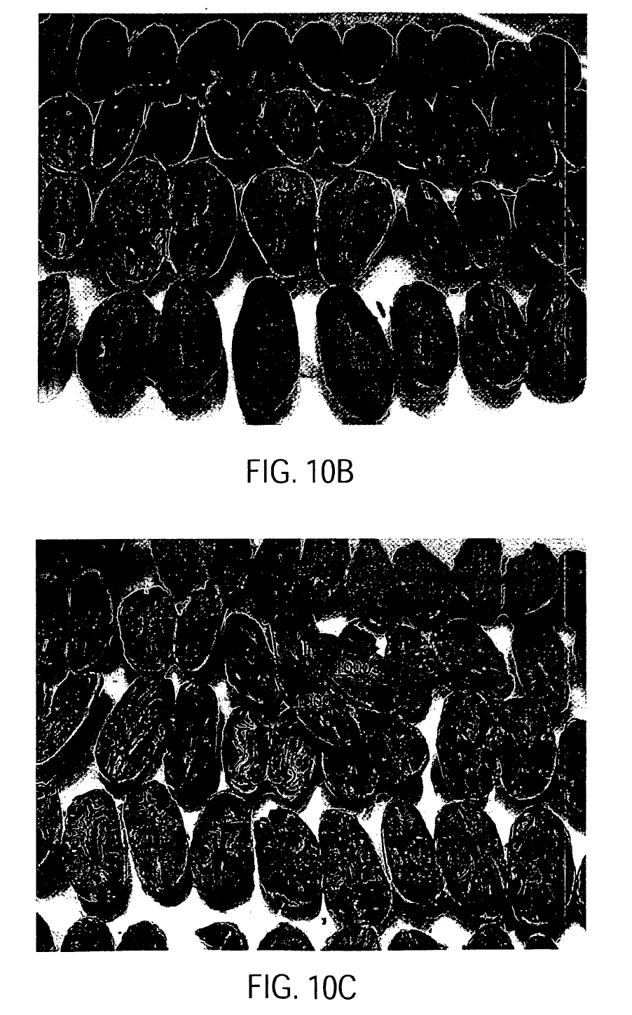

FIG. 10A is a photograph of cocoa bean cotyledons in an early stage of aeration 138, after fermentation 122, condensation 130, and piercing 134. FIG. 10B is a photograph of cocoa bean cotyledons in a later stage of aeration 138. FIG. 10C is a photograph of dry, green cocoa bean cotyledons (nibs in the shell) that have been fermented, condensed, pierced, and fully aerated 138. Cocoa bean 1000, which appears purplish in contrast to the other beans, was fermented, condensed, and then aerated without piercing for comparison.

As seen by the lighter color in the middle of the cotyledons in FIG. 10A progressing toward the uniform dark color in FIG. 10C, aerating (drying) of pierced seeds proceeds inwardly from the shell toward the interior of the seeds. This drying front progression, from the shell toward the interior of the seeds, allows a slow, controlled wicking, resulting in homogeneous dry, green beans. In contrast, drying of cocoa beans that have been partially deshelled by opening via scoring, scraping, cracking, crushing, and/or winnowing may proceed from the interior of the beans toward the exterior of the bean (outer cotyledon layers) proximate the shell. This drying front progression from interior to exterior may not allow the controlled wicking demonstrated for pierced beans.

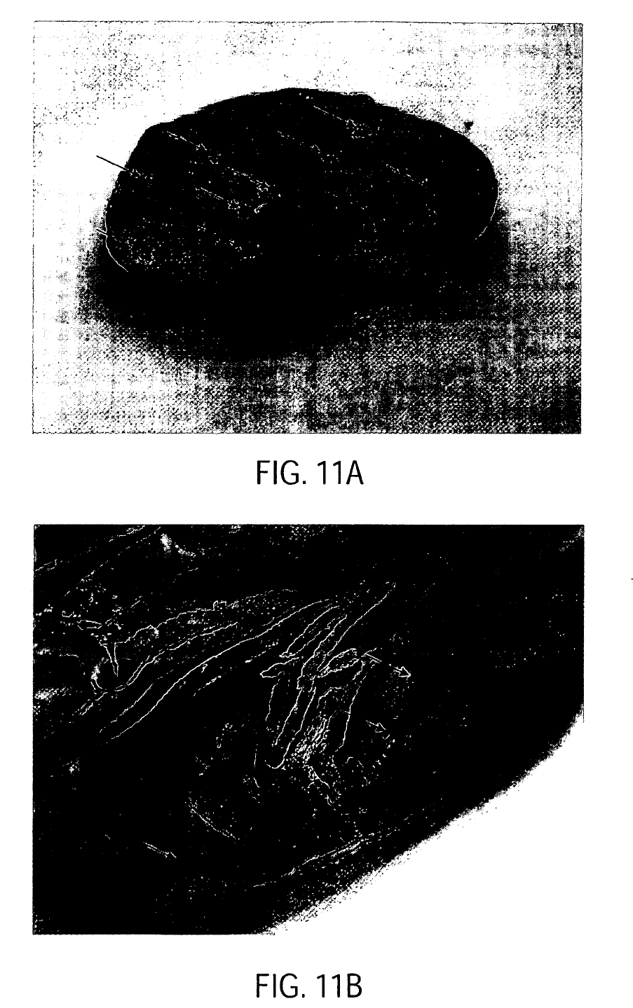

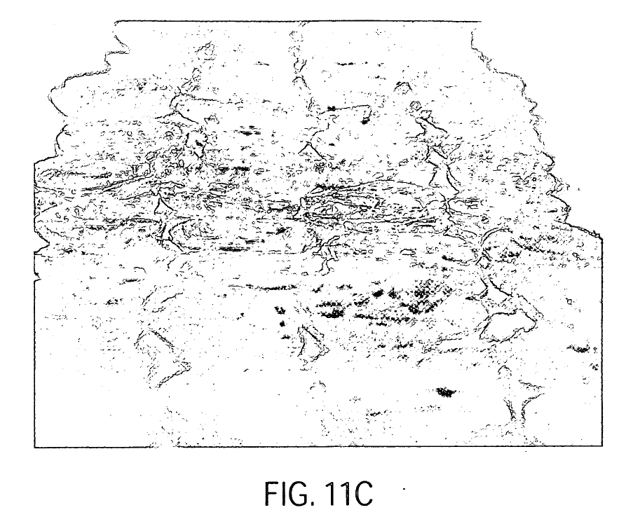

FIG. 11A is a photograph showing a pierced, intact, dry, green cocoa bean after fermentation 122, aeration 138, and dehydration 142. FIG. 11B is a photograph of a cross-sectional view of a pierced, dry, green cocoa bean after fermentation 122, aeration 138, and dehydration 142. Arrows indicate openings in the shell and cotyledons. FIG. 11C is a photograph showing cotyledons of pierced, dry, green cocoa beans after dehydration 142. The cotyledons exhibit substantially uniform brown coloration and do not exhibit defects such as slaty beans or purple coloration.

The following examples are provided to more fully illustrate some of the embodiments of the present invention. It should be appreciated by those of skill in the art that the techniques disclosed in the examples which follow represent techniques discovered by the inventors to function well in the practice of the invention, and thus can be considered to constitute exemplary modes for its practice. However, those of skill in the art should, in light of the present disclosure, appreciate that many changes can be made in the specific embodiments that are disclosed and still obtain a like or similar result without departing from the spirit and scope of the invention.

### EXAMPLE 1

Selected cacao fruits with high fruit pulp content and high soluble solid contents of the pulp were received in a central processing facility with cement floor and roof. These fruits were washed and surface sterilized as a preparatory step prior to pod opening. Cleaned cacao fruits were opened by clean, gloved hands using a sharp clean knife with a carbon steel blade of 25 cm length, 4.5 cm height, and 2 mm width. Fruits were broken in two parts, roughly across the middle of the husk, to open the fruit interior containing the sweet juice and pulp-surrounded wet seeds adhering to the central placental material. Opened fruits were visually inspected for signs of disease or spoiling. Roughly 5% of fruits were rejected at this point and discarded. The selected opened fruits, many times including the basal half of the fruit husk, seeds with adhering fruit pulp and juice, and placenta, were placed in a clean, round, aluminum basin (diameter 70 cm and depth 15 cm). Seeds were manually separated from husk and placenta, and adhering clusters of seeds were separated. The husk and placenta were discarded and the wet seeds were accumulated in the basin. A 15 L graduated inox steel bucket was filled with wet cacao seeds to determine wet cacao volume. The bucket was weighed on a two-beam balance (15 kg max. capacity, Cauduro Ltda. Cachoeira do 5 ul, RS Brasil).

Wet cacao prepared as described above was placed in wooden sweat boxes (95 cm width, 91 cm depth, 53 cm height, with 20 cm wide boards with 5 mm spacing between boards for sweating exit and aeration). The wet cacao was then box fermented using an insolated cover (polystyrene, 25 mm thickness) for optimal thermal generation during fermentation. The temperature of fermenting mass increased to over 50° C. by day five of fermentation, and most seeds had plumped as well by that same time. Fermented seeds were spread on a wooden drying platform and turned at regular intervals throughout the drying. After several hours on the drying platform, approximately 500 moist, wet seeds were manually pierced with an inox sewing needle of 0.5 mm diameter and 4 cm length. Seeds were placed on a wooden platform and secured between thumb and forefinger and pierced 10 to 15 times, then rotated 180 degrees and pierced on the opposite side another 10 to 15 times for a total of between 20 and 30 piercings per seed. The piercing was carried out in such a way that the needle pierced the shell in a first location, the cotyledons, and the shell in a second location before contacting the wooden surface. Piercings were evenly distributed around the seed surface. Pierced seeds then were returned to the wooden drying platform and sun dried for five days. All pierced seeds showed excellent browning during drying. Significant browning of pierced seeds was visually noted after 12 and 24 hours. Browning of pierced seeds appeared complete to the naked eye in all seeds after 48 hours on the drying platform. Pierced seeds showed far superior browning than non-pierced seeds, and dried more quickly and to a lower moisture content than non-pierced seeds. A small sample of excellent quality dry green cocoa beans was produced.

### EXAMPLE 2

Wet cacao prepared as described in EXAMPLE 1 underwent an alcoholic fermentation in a sealed, cylindrical food-safe container of 68 cm height and 33 cm diameter (approximately 75 L volume), similar to the embodiment depicted in FIG. 2. The sealed container in this example was equipped with an air lock to release gas from the container. Approximately 60 L fresh wet cacao seed with juice and pulp were fermented in the container. Alcoholic fermentation progressed with noticeable production of alcohol in the liquid medium, and production of CO₂ gas that rose through the fermenting liquid and exited through the vapor lock. Fermentation proceeded for eight days until seeds began to plump. Plumping continued to occur in relatively more of the fermenting seeds (noticed by repeated sampling of fermenting seeds), until all seeds taken from a sample of 1 L at twelve days were shown to have plumped. Temperature of fermenting mass ranged from 24 to 28° C. throughout the fermentation process, and the temperature did not vary significantly from the mass interior to the mass exterior (proximate the container wall). At fourteen days under alcoholic fermentation, seeds were removed from the fermentation container.

Sample 1. A first sample of 2 L of wet fermented seeds and fermented pulp was removed. The fermented pulp was manually depulped by removing seeds one by one from the mass and scraping any adhering pulp from the seeds by hand. These manually depulped, fermented, wet seeds were pierced 20 to 30 times each as described in EXAMPLE 1. The remainder of the fermented seeds with fermented pulp were removed from the fermentation container, spread on a wooden drying platform, turned at regular intervals as described in EXAMPLE 1.

Sample 2. A second sample of approximately 1000 seeds was taken from the drying platform after some hours of drying. These seeds exhibited condensed, fermented pulp on the shell exteriors and moist and wet interiors. Seeds from Sample 2 were pierced manually, as described in EXAMPLE 1, 20 to 30 times per seed. Significant browning was noted 12 hours after piercing in both Sample 1 (manually depulped) and Sample 2 (condensed pulp). All pierced seeds browned to a high degree and dried readily. Pierced seeds (both depulped and condensed) produced excellent quality dry, green cocoa beans.

### EXAMPLE 3

Wet cacao prepared as described in EXAMPLE 1 underwent an alcoholic fermentation as described in EXAMPLE 2. After two weeks of alcoholic fermentation, fermented seeds and pulp (approximately 120 L total) were removed from two containers. About 4 to 6 L of wet, fermented seeds were spread on trays of woven aluminum wiring (0.7 mm diameter) and placed in an dryer similar to the embodiment depicted in FIG. 3. Condensation began by convective air flow at ambient temperature and relative humidity for 24 hours. Trays were removed and seeds and pulp were mixed to increase condensation and decrease adhesion of seeds to each other. After approximately 24 hours at ambient temperature, the temperature was increased to 45° C. to promote condensation of fermented pulp onto seed shell exteriors. Trays were removed and rotated 180° to effectively reverse the direction of airflow over the seeds, with or without mixing of seeds and spreading on trays, and replaced in a shuffling fashion to the dryer to allow seeds to aerate evenly at the top, middle, and bottom of the vertical stack of 20 trays as they were positioned in the cabinet. After 6 to 8 hours, condensation had occurred such that the seeds were tacky wet to slightly dry to the touch at the shell surface, while still moist and wet with purple interiors when cut open. Seeds with condensed pulp were run through a perforating machine, similar to the embodiment shown in FIG. 6, from one to four times and repositioned on trays and dried to an estimated 5 to 7 wt % moisture content at 60° C. for 24 hours. During aeration, seeds were mixed and repositioned on trays or the trays were rotated 180° at two hour intervals until dry. Dried green cocoa beans of excellent quality and browning were produced. All pierced seeds showed browning, while the extent of browning was positively correlated and the amount of purple pigment remaining in the dry seed was negatively correlated with the number of passes through the perforation machine.

### EXAMPLE 4

Wet cacao prepared as described in EXAMPLE 1 underwent an alcoholic fermentation as described in EXAMPLE 2. After four weeks of alcoholic fermentation, fermented seeds and pulp from two fermentation containers (approximately 120 L) were removed from the containers. About 4 to 6 L of wet fermented seeds were spread on trays and placed in the dryer. Condensation of pulp to the shell exterior occurred at 45° C. with mixing of seeds on trays described in EXAMPLE 3 to improve aeration and reduce adhesion of seeds to each other. After 4 to 8 hours, the shells of the seeds were tacky wet, moist, or slightly dry to the touch, while the interiors remained wet and moist. Condensed seeds (i.e., seeds with pulp condensed on the shells) were passed through the perforating machine 4 times (producing an average 12.7 piercings per bean), spread on the trays, and returned to the dryer or spread out on a traditional wooden drying platform. Aeration of perforated seeds in a dryer over six days occurred at ambient temperature (21 to 30° C.) and relative humidity (95 to 70%). Perforated, aerated seeds were dried at 60° C. for 12 hours until an estimated moisture content of 5 to 7 wt % was reached. Dry, green cocoa beans of excellent quality were produced from wooden platform and dryer-dried seeds. All cocoa beans browned to a high degree and had pleasant aroma and good texture.

### EXAMPLE 5

Wet cacao prepared as described in EXAMPLE 1 underwent an alcoholic fermentation as described in EXAMPLE 2. After four months of alcoholic fermentation, fermented seeds and pulp from two fermentation containers (approximately 120 L) were removed from the containers. About 4-6 L of wet fermented seeds were spread on trays and placed in the dryer. Condensation of pulp to the shell exterior occurred at 42° C. with mixing of seeds on trays as described in EXAMPLE 3 to improve condensation and reduce adhesion of seeds to one another. After 6 to 9 hours, the shells of the seeds were tacky wet, moist, or slightly dry to the touch, while the interiors remained wet and moist. Condensed seeds were passed through the perforating machine three or four times, spread on the trays, and returned to the dryer for aeration at ambient temperature (23 to 36° C.) and relative humidity for two weeks until dried (estimated moisture content 6-8 wt %). Cocoa beans were given an additional one hour drying at 60° C. to ensure good keeping quality. Dry, green cocoa beans of excellent and consistent browning and pleasant aroma were obtained.

### EXAMPLE 6

Wet cacao prepared as described in EXAMPLE 1 was placed in 4 L freezer safe plastic food storage bags, the air evacuated, and the bags sealed with a heat strip. Bagged wet cacao was frozen at −20° C. Frozen wet cacao was thawed and underwent an alcoholic fermentation as described in EXAMPLE 2. After three weeks of alcoholic fermentation, fermented seeds and pulp from the fermentation container (approximately 32 L) were removed from the container. About 4-6 L of wet, fermented seeds were spread on trays and placed in the dryer. Condensation of pulp to the shell exterior occurred at 42° C. with mixing of seeds on trays as described in EXAMPLE 3 to improve condensation and reduce adhesion of seeds to one another. After 4 to 8 hours, the shells of the seeds were tacky wet, moist, or slightly dry to the touch, while the interiors remained wet and moist. Condensed seeds were passed through the perforating machine three or four times, spread onto the trays, and returned to the dryer to aerate at ambient temperature and relative humidity for two days. After 48 hours of aeration, the seeds were dried at 60° C. for 6 hours until the moisture content was estimated to be about 5-7 wt %. Good dry, green cocoa beans were produced with nice browning and pleasant aroma.

### EXAMPLE 7

Wet cacao prepared as described in EXAMPLE 1 underwent an alcoholic fermentation as described in EXAMPLE 2. After twelve months of alcoholic fermentation, fermented seeds and pulp from a fermentation container (approximately 45 L) were removed from the container. About 4 L of wet, fermented seeds were spread on trays and placed in the dryer. Condensation of pulp to the shell exterior occurred at 45° C. with mixing of seeds on trays as described in EXAMPLE 3 above to improve condensation and limit adhesion of seeds to one another. After 8 to 12 hours, the shells of the seeds were tacky wet, moist, or slightly dry to the touch, while the interiors remained wet and moist. Condensed seeds were passed through the perforating machine four times, spread onto the trays, returned to the dryer to aerate at 50° C. for 12 hours. The seeds were then aerated at ambient temperature (21 to 24° C.) for 12 hours, then dried at 60° C. until dry. The moisture content was estimated to be about 6 to 8 wt %). Dry, green cocoa beans were produced having good browning and highly ammonia-like aroma.

### EXAMPLE 8

Wet cupuaçu (Theobroma grandiflora) was prepared similarly as described for cacao in EXAMPLE 1. Approximately 200 L of wet cupuaçu, including pulp, juice, and seeds, was placed in a 240 L fermentation container. The fruit juice and pulp underwent an alcoholic fermentation as described in EXAMPLE 2. Seeds plumped beginning at day 7 and all 20 seeds taken from a sample of after 14 days were plumped. After approximately 4 months, a sample of approximately 30 L of fermented cupuaçu juice, pulp, and seeds was taken from the fermentation container. Seeds were removed from the fermented juice and pulp and set on a drying platform, as in Sample 1 of EXAMPLE 2, as well as set on trays as in Sample 2 of EXAMPLE 2. Seeds were allowed to become tacky wet on both the drying platform and the dryer and were then perforated manually 20 to 30 times per seed with an inox needle (diameter of 0.7 mm). The seeds were returned to the drying platform or dryer. Seeds on drying platform dried by 3 days and showed excellent and consistent browning (a light golden brown) and good aroma in all pierced seeds when examined upon cutting the seeds in half to exhibit the seed interior. Pierced seeds aerated at ambient temperature in the dryer produced a highly aromatic and pleasant scent that had pronounced and distinct floral and sweet citrus-like notes. Pierced seeds taken from the dryer showed 100% browning of the seed interiors (a light golden brown) upon examination after splitting in half with a knife after 24 to 48 hours aeration. After 4 days of aeration at ambient temperature, the temperature was raised to about 50° C. for 4 hours to dry the beans to a stable moisture content. Dry, green cupuaçu beans of excellent quality were produced with consistent and complete browning and pleasant aroma.

### EXAMPLE 9

Wet cacao was prepared as described in EXAMPLE 1. A non-fermented sample (Sample 1) of approximately 6 L was depulped manually by placing approximately 300-400 ml wet fermented seeds and pulp in a cylindrical plastic sieve having a mesh screen on bottom and side (20 cm diameter by 9.5 cm height, 3.0 mm diameter of plastic wires and distributed at 65 mm intervals from wire center). Depulping was done by hand using vigorous back and forth and circular agitating motions of the sieve basket for between 15 and 45 seconds per 300-400 ml load. After depulping, the seeds were placed in a nine-tray (15 square feet of total tray area) food dehydrator with thermostatically controlled electric heating element (Excalibur 3000, Model #4926T220, Excalibur Products; Sacramento, Calif., USA) at density of between 300-600 ml seeds per tray. Seeds were dried under pulsed convective airflow at 33° C. with pulses to 44° C., with daily periodic mixing of seeds, for 5 days to achieve a stable moisture content of approximately 4-6 wt %.

Active dried wine yeast, Saccharomyces cerevisiae UCD 522, MAURIVIN™ (manufactured by Mauri Yeast Australia Pty Ltd.; Toowoomba, Queensland, Australia) was added to the fresh wet cocoa while in the aluminum basin (described above) at the rate of 1 level measuring teaspoon per 20 L wet cocoa. Wet cocoa underwent alcoholic fermentation as described in EXAMPLE 2. In this case, after placing the wet cocoa into the fermentation container, the container was first covered with a cloth to promote aerobic fermentation, and then, after 24 to 48 hours, the container was sealed with a lid having an airlock. After 7, 14, 18 and 31 days of alcoholic fermentation, fermented seeds and pulp from two fermentation containers (each containing approximately 40-50 L) were removed from the container. The two 7-day fermentation samples were removed from the fermenting containers and condensed over the course of three days at ambient with periodic mixing of seeds and rotating of trays. Two brief pulses (20 and 40 minutes) of heat at 40-45° C. were given in the evening to promote the condensation of pulp and produce tacky wet seeds. The 14-, 18-, 31-day fermentation samples underwent depulping in 300-400 ml batches in the sieve basket and were condensed as described in EXAMPLE 3 at ambient temperature (20° C.-28° C.). After approximately 24 hours of condensation, the seeds were tacky wet, moist, or slightly dry to the touch, while the interiors remained wet and moist. A 7 kg (dry weight) sample of condensed seeds was loaded into the Excalibur 3000 food dehydrator and dried between about 33° C.-35° C. with thermostatically controlled repeated pulses of 44° C.-46° C. for 4 days to a stable moisture content of 4-5 wt %. The remaining condensed seeds were passed through the perforating machine two times, spread evenly onto the trays, and returned to the dryer to aerate at ambient temperature (20° C.-28° C.) for two-six days with periodic, daily 180° rotation of the trays and mixing of seeds on trays. Perforated and aerated seeds were then loaded into the Excalibur 3000 food dehydrator at loading densities ranging from 7 to 10.5 kg of dried seeds per batch and dried at between 33-35° C. with thermostatically controlled repeated pulses of 44-46° C. for 24 hours to a stable moisture content of 4-6 wt %. Dried cocoa was stored in plastic food bags sealed with twist ties and placed in an airtight container similar to that used for fermentation.

SAMPLE PREPARATION. Samples of fermented, dried cocoa were prepared for as described below for analytical analysis. Cotyledon material was prepared by deshelling removing the embryo and root radical from approximately 50-100 seeds. Deshelled and degermed cotyledons were ground for 2-4 minutes in a hand-held 250 W electric mini food processor (Walita Mix, model R11353, Philips do Brasil Ltda, Division Walita; Varginha, MG, Brazil) until nib pieces of no greater than approximately 3 mm remained in the sample. Samples were then further ground to a fine powder with a ceramic mortar and pestle and passed through a 42 mesh sieve onto wax paper. Ground, sieved samples were stored in plastic bags at ambient conditions.

SAMPLE ANALYSIS—pH. 10 g ground and sieved (42 mesh) cocoa cotyledon was placed in a 300 ml beaker. Boiling deionized water (90 ml) was added to the 300 ml beaker while stirring with a glass rod to create a 10% wt/volume slurry. The slurry was stirred for 10 seconds, the stirring rod was removed, and the beaker was placed in an ice bath and cooled to 23-26° C., allowing the dispersed solids to settle. After settling of the particulate matter, 50 ml of the supernatant was decanted into a 50 ml graduated cylinder and immediately transferred to a 100 ml beaker. The sample pH was determined by immersing the electrode of a pH meter into the supernatant under constant stirring with a magnetic stir bar.

SAMPLE ANALYSIS—Titratable Acidity. Sample titratable acidity was obtained from the sample used for pH. Immediately after pH determination, the 50 ml of solution was titrated to pH 8.1 with 0.1N NaOH added dropwise from a 50 ml graduated burette. Titratable acidity (ml 0.1N NaOH per g sample) was calculated using 5 g (50 ml) as the sample mass.

SAMPLE ANALYSIS—Fermentation Index. 0.5 g ground and sieved (42 mesh) cocoa cotyledon was placed in a 100 ml glass flask. 50 ml methanol:HCl (97:3) solution was added to the flask. The flask was covered and the mixture set in a dark refrigerator at 6° C. for 18 hours. The mixture was then vacuum filtered and 300 ml of the filtered extract was transferred by pipette into three wells of a microplate. The absorbances of the extract at 460 nm and 530 nm were read using a VERSAMAX™ Microplate Reader with Softmax ProSoftware-1993-2006 (Molecular Devices Corp., Sunnyvale, Calif., USA). Absorbance readings were taken in triplicate. The fermentation index was reported by taking the mathematical mean of the fermentation indices calculated from the three readings.

SAMPLE ANALYSIS Cut—Test/Fermentation Factor. A cut test is a standard procedure for assessing quality of cocoa beans. Fermentation factor, calculated from the visual assessment of cut cocoa beans, is a numerical representation of the level of fermentation of the sample. Samples of dried cocoa beans were cut in half lengthwise, visually inspected for color as well as defects, and divided into four categories according to the color of the exposed cut surfaces of the cotyledons.

To determine color, the halved beans were placed on a white surface and exposed to bright but indirect sunlight near a window in a white room and inspected by eye for visual color appearance. Four fermentation categories were determined based on the visual appearance of color of the exposed cut surfaces of the cotyledons: 1) slaty (non-fermented or very under-fermented beans); 2) purple (under-fermented); 3) purple/brown (partially fermented); and 4) brown (well-fermented). Purple/brown cocoa beans are those that have portions of the cut surfaces of the cotyledons with both purple/violet and brown color seen either in patches or diffusely distributed along the cut surfaces. Cut beans were separated into the four fermentation categories. Each category was assigned a value of 1 (slaty), 2 (purple), 0.3 (purple/brown), or 4 (brown). The percentage of beans from a sample that comprised each category was multiplied by the color value corresponding to each category, and the products were summed to yield the fermentation factor for the sample.

Fermentation factor was calculated in triplicate for each fermentation treatment by cutting, visually inspecting and categorizing 150 beans and calculating a fermentation factor. Fermentation factor, which can range from 100 (100% slaty beans) to 400 (100% brown beans) was reported for each sample as the mathematical mean of three independently calculated fermentation factor values from 50 beans. For example, the fermentation factor for a sample of 50 beans scored as 0: slaty, 9: purple, 30: purple/brown, and 11: brown is: ((0*1)+(18*2)+(60*3)+(22*4))=304.

TABLE 1 shows average fermentation index (FI), pH, titratable acidity (TA), and fermentation factor (FF) for 23 cocoa seed samples that underwent alcoholic fermentation 122 (Ferm.) from 0 (unfermented) to 31 days, with aeration 138 (Aer.) ranging from 0 (no aeration) to 6 days. Perforation 134 of the seeds (Perf.) is indicated by “no” (unperforated) or “yes” (perforated). Titratable acidity is given as ml of 0.1 N NaOH per gram of sample. Average absorbance of the samples at 460 nm (A(460)) and 530 nm (A(530)) used to calculate FI are also listed.

TABLE 1

| | Ferm. | | Aer. | | A | A | | | |
|---|---|---|---|---|---|---|---|---|---|
| Sample | (days) | Perf. | (days) | Avg. FI | (460) | (530) | pH | TA (ml/g) | FF |
| 1 | 0 | no | 0 | 0.342 | 0.472 | 1.379 | 6.52 | 0.560 | 200 |
| 2 | 7 | no | 4 | 0.701 | 0.423 | 0.603 | 5.55 | 0.900 | 302 |
| 3 | 7 | yes | 0 | 0.697 | 0.350 | 0.502 | 5.65 | 0.880 | 400 |
| 4 | 7 | yes | 2 | 0.691 | 0.314 | 0.454 | 5.77 | 0.760 | 400 |
| 5 | 7 | yes | 3 | 0.759 | 0.323 | 0.425 | 6.01 | 0.640 | 400 |
| 6 | 7 | yes | 4 | 0.772 | 0.310 | 0.402 | 5.92 | 0.733 | 400 |
| 7 | 7 | yes | 5 | 0.738 | 0.283 | 0.384 | 5.84 | 0.833 | 400 |
| 8 | 14 | no | 0 | 0.691 | 0.426 | 0.616 | 5.59 | 1.280 | 306 |
| 9 | 14 | yes | 3 | 0.862 | 0.412 | 0.478 | 5.76 | 0.837 | 400 |
| 10 | 14 | yes | 4 | 0.835 | 0.263 | 0.315 | 5.90 | 0.820 | 400 |
| 11 | 14 | yes | 4 | 0.785 | 0.271 | 0.346 | 5.79 | 0.814 | 400 |
| 12 | 14 | yes | 5 | 0.795 | 0.290 | 0.365 | 5.91 | 0.778 | 400 |
| 13 | 18 | no | 0 | 0.763 | 0.455 | 0.596 | 5.28 | 1.600 | 300 |
| 14 | 18 | yes | 2 | 0.827 | 0.282 | 0.341 | 5.47 | 1.020 | 400 |
| 15 | 18 | yes | 3 | 0.821 | 0.265 | 0.323 | 5.50 | 1.023 | 400 |
| 16 | 18 | yes | 4 | 0.859 | 0.271 | 0.315 | 5.52 | 1.087 | 400 |
| 17 | 18 | yes | 6 | 0.798 | 0.274 | 0.344 | 5.29 | 1.200 | 400 |
| 18 | 31 | no | 3 | 1.063 | 0.403 | 0.379 | 4.37 | 1.460 | 332 |
| 19 | 31 | yes | 2 | 1.012 | 0.350 | 0.346 | 4.53 | 1.200 | 400 |
| 20 | 31 | yes | 3 | 1.007 | 0.351 | 0.348 | 4.43 | 1.360 | 400 |
| 21 | 31 | yes | 3 | 0.969 | 0.338 | 0.349 | 5.15 | 1.044 | 400 |
| 22 | 31 | yes | 4 | 1.043 | 0.358 | 0.343 | 5.15 | 1.167 | 400 |
| 23 | 31 | yes | 5 | 0.981 | 0.322 | 0.329 | 4.63 | 1.128 | 400 |

The oxygen radical absorbance for Sample 12 (Table 1), expressed as a micromole Trolox equivalent (TE) per gram of sample, was found to be 439 (water-soluble antioxidant capacity) and 4 (lipid-soluble antioxidant capacity), for a total oxygen radical absorbance of 443 μmole TE/g.

An ammonia content of various samples in Table 1 is less than 500 ppm, less than 100 ppm, or, in some cases, less than 50 ppm.

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating seeds, the method comprising
piercing a multiplicity of seeds such that shells of a majority of the seeds are pierced;
aerating the pierced seeds; and
reducing a water content of the pierced seeds.

2. The method of claim 1, wherein the seeds comprise cocoa beans.

3. The method of claim 1, wherein the majority of the seeds are unfermented at the time of piercing.

4. The method of claim 1, wherein piercing the seeds comprises forming an opening in each shell of the majority of the seeds.

5. The method of claim 4, wherein each opening has an opening area of between about 0.5 and 15 mm².

6. The method of claim 1, wherein piercing the multiplicity of seeds comprises forming an opening in a shell and a cotyledon of the majority of the seeds.

7. The method of claim 1, wherein piercing the multiplicity of seeds comprises inserting one or more needles in the majority of the seeds.

8. The method of claim 1, further comprising fermenting the multiplicity of seeds.

9. The method of claim 8, wherein the fermented, dry seeds are fermented, dry cocoa beans.

10. The method of claim 1, further comprising curing the multiplicity of seeds.

11. The method of claim 10, wherein piercing the multiplicity of seeds occurs before curing the multiplicity of seeds.

12. The method of claim 1, further comprising roasting the pierced seeds.

13. A method of treating seeds, the method comprising
placing a bulk quantity of seeds in a container;
forming a mass in the container, wherein the mass comprises the bulk quantity of seeds and liquid;
sealing the container to create a substantially closed environment inside the container; and
fermenting the mass in the sealed container.

14. The method of claim 13, wherein the seeds comprise cocoa beans.

15. The method of claim 13, wherein fermenting the mass comprises alcoholic fermentation.

16. The method of claim 13, further comprising piercing the bulk quantity of seeds.

17. The method of claim 13, further comprising piercing the seeds before placing the seeds in the container.

18. The method of claim 13, further comprising monitoring a temperature within the sealed container.

19. The method of claim 13, further comprising controlling a pressure inside the sealed container.

20. The method of claim 13, further comprising controlling a pH of the liquid.

21. The method of claim 13, further comprising reducing water content of the fermented mass to produce a bulk quantity of fermented, dry seeds.

22. The method of claim 21, wherein the fermented, dry seeds are fermented, dry cocoa beans.

23. A method of treating seeds, the method comprising
placing a multiplicity of pierced seeds in a ventilated enclosure;
forcing air through the enclosure such that the seeds are exposed to the air; and
mixing the seeds.

24. The method of claim 23, wherein the seeds comprise cocoa beans.

25. The method of claim 23, further comprising monitoring a temperature of the air.

26. The method of claim 23, further comprising monitoring a temperature inside the enclosure.

27. The method of claim 23, further comprising monitoring a relative humidity inside the enclosure.

28. The method of claim 23, further comprising reversing a direction of the forced air.

29. The method of claim 23, wherein a majority of the seeds are pierced in one or more locations.

30. A bulk quantity of treated seeds, in which
a majority of the treated seeds have pierced shells; and
an average water content of the treated seeds is less than about 10 wt %.
